(19) United States
(12) Patent Application Publication
(10) Pub. No.: US 2017/0224775 A1
(43) Pub. Date: Aug. 10, 2017

(54) Lv PEPTIDE, ANTI-Lv ANTIBODY AND METHODS THEREOF

(71) Applicant: The Texas A&M University System, College Station, TX (US)

(72) Inventors: Gladys Ko, College Station, TX (US); Lih Kuo, Temple, TX (US)

(21) Appl. No.: 15/424,816

(22) Filed: Feb. 4, 2017

Related U.S. Application Data
(60) Provisional application No. 62/291,283, filed on Feb. 4, 2016.

(51) Int. Cl. A61K 38/18; C07K 16/22

(52) U.S. Cl. CPC A61K 38/1891; C07K 16/22; C07K 2317/76; A61K 2039/505

(57) ABSTRACT

Embodiments of the invention are directed to the administration of the composition containing a portion of the peptide Lv to a subject to promote angiogenesis. The interaction between peptide Lv and VEGFR2 represents a novel pathway regulating angiogenesis and cardiac function. The artificially modified peptide Lv, the inverso D-peptide Lv, shows a similar efficacy in promoting endothelial cell proliferation as the natural peptide Lv. Additional embodiments are directed to the use of anti-Lv antibodies for reducing angiogenesis and dampening L-VGCC activities.

### CROSS-REFERENCE TO RELATED APPLICATIONS

This application claims the benefit of priority to and incorporates by reference the entire disclosure of U.S. Provisional Patent Application No. 62/291,283 filed on Feb. 4, 2016.

### STATEMENT REGARDING FEDERALLY SPONSORED RESEARCH AND DEVELOPMENT

This invention was made by Government support under contracts R01EY017452 and R21EY023339 awarded by The National Eye Institute of the National Institutes of Health. The Government has certain rights in the invention.

### BACKGROUND OF THE INVENTION

Peptide Lv is a bioactive peptide that was identified through bioinformatics screening. Peptide Lv is a short peptide with only 40 amino acids in humans (49 amino acids in mice) and a ˜5.8 kD molecular weight. It can be applied to various biomedical processes that involve new blood vessel formation (angiogenesis), such as, for example, wound healing and cardiovascular repairing after ischemia injuries and/or disease insults. It can also be used for forming new lymphatic vessels (lymphangiogenesis) and enhancing immunity, metabolism, and homeostasis. The molecular mechanism of peptide Lv on angiogenesis and lymphangiogenesis is that peptide Lv is able to activate vascular endothelial growth factor (VEGF) receptors and promote proliferation of endothelial cells that are required for angiogenesis, lymphangiogenesis, and cardiovascular repairing. Peptide Lv is like VEGF and is able to dilate blood vessels as a vasodilator, and thus it can be used to enhance cardiovascular function, decrease blood pressure, or treat microvascular insufficiency associated with coronary artery diseases. In addition, peptide Lv is able to enhance physiological functions of neurons and cardiomyocytes by augmenting the L-type voltage-gated calcium channel (L-VGCC) currents, since L-VGCCs are essential in the contraction of cardiomyocytes and neurotransmitter release from neurons. Therefore, peptide Lv can be used to enhance cardiac contraction, cardiac output, and neural function.

The antibody specific against peptide Lv (anti-peptide Lv antibody) can specifically block the effect of peptide Lv, VEGF, or VEGF receptor activation. Anti-peptide Lv antibody can be used to stop diseases that have pathological angiogenesis and lymphangiogenesis or L-VGCC hyperfunction, such as cancers, diabetic retinopathy, age-related macular degeneration, cardiac arrhythmia, various neuropathy, and others.

The main peptide Lv coding region contains 49 amino acids in mice and 40 in human, which is conserved across human, mouse, rat, and chicken. The peptide Lv mRNA is widely expressed in various organs including the lung, spleen, intestine, retina, and various regions in the brain. However, the binding partners and receptors of peptide Lv are unknown, which completely cloud the physiological and pathological functions of peptide Lv. Since the brain expresses peptide Lv, a study applied a proteomics approach using a mouse whole brain preparation, and identified that vascular endothelial growth factor receptor 2 (VEGFR2) is a binding partner of peptide Lv. VEGFR2 (KDR/FLK-1) belongs to a tyrosine kinase receptor family that is subjected to autophosphorylation of tyrosine residues after the receptor is activated via ligand binding. VEGFR signaling is essential for the development of the cardiovascular system during embryonic stages, and it is critical for angiogenesis, lymphangiogenesis, vasodilation, and wound healing throughout adulthood. There are four major tyrosine autophosphorylation sites (951, 1054/1059, 1175, and 1212/1214) located in the intracellular domain of VEGFR2. These four phosphorylation sites may elicit different signaling pathways upon activation, thus prompting different functions. Since VEGFR2 is important in the developing heart, the interaction between peptide Lv and VEGFR2 in the embryonic chicken heart was further verified. The physiological effects of peptide Lv in embryonic chicken cardiomyocytes and human endothelial cells was verified, and a potential functional link between peptide Lv and VEGFR2 was found.

It has been shown that exogenous peptide Lv increases L-VGCC currents in cultured retinal photoreceptors and cardiomyocytes. The augmentation of L-VGCC currents is mainly through the increase of mRNA and protein expressions of L-VGCCα1 subunits. The L-VGCCs are highly expressed in the nervous and cardiovascular systems. The voltage dependent calcium entry through L-VGCCs promotes various cellular processes including regulating intracellular calcium homeostasis, modulating activities of calcium-dependent enzymes, and regulating gene expressions. In particular, L-VGCCs are essential for neurotransmitter release from non-spiking neurons, such as photoreceptors and bipolar cells in the retina. In brain neurons, L-VGCCs mediate activity-dependent calcium influx, so L-VGCCs are critical in gating neuronal activities. While L-VGCCs are required for cognitive functions, learning and memory, dysregulation or hyperactivity of L-VGCCs causes epilepsy and aging-related memory loss. In cardiomyocytes, the L-VGCCs contribute to the action potential and excitation-contraction coupling, and dysregulation of L-VGCCs in the cardiovascular system causes cardiac arrhythmias and heart failure. Since L-VGCCs are important for the function of cardiomyocytes, it was explored that peptide Lv regulated cardiomyocyte physiology by augmenting the expression of L-VGCCs in part through VEGFR2-dependent signaling.

Peptide Lv can be used to promote: neurological function, vascular function, cardiac contractility and output, tissue repair and wound healing, through regulating L-VGCCs, angiogenesis and lymphoangiogenesis, and neovascularization, while anti-peptide Lv antibody antagonizes those functions. Peptide Lv can also be used to reduce hypertension and improve blood flow to the ischemic tissues that suffer from microvascular spasm or occlusion through vasodilation. By augmenting L-VGCCs, peptide Lv can enhance/promote neural and cardiac electrical transmission for treatment of neural/cardiovascular diseases and heart failure related to the conduction deficiency. Anti-peptide Lv antibody can be used in treating cancers, diabetic retinopathy, age-related macular degeneration, and other diseases that are related to pathological angiogenesis/lymphangiogenesis or L-VGCC hyperfunctions, such as cardiac arrhythmia, and various neural dysfunction and neuropathy (e.g. epileptic disorders, age-dependent memory deficits).

There are many diseases and conditions that are involved angiogenesis, lymphangiogenesis, or L-VGCC disorders. For example, wound healing and cardiovascular repairing require angiogenesis and lymphangiogenesis, so peptide Lv can enhance these necessary biological processes. The deficiency in cardiac L-VGCC can lead to heart failure and certain forms of arrhythmia, so peptide Lv will be able to treat/prevent these diseases. Pathological angiogenesis, lymphangiogenesis and L-VGCC hyperfunction take place in many diseases such as cancer, diabetic retinopathy, age-related macular degeneration, cardiac tachycardia and various neural dysfunction and neuropathy (epileptic disorders, age-dependent memory deficits) and many more. Thus, anti-peptide Lv antibody can be used to stop or dampen pathological angiogenesis, lymphangiogenesis, and neural dysfunction. Therefore, both peptide Lv and anti-peptide Lv antibody will have great potential for treating various medical conditions.

Applications related to VEGF and VEGF receptors are generally known. However, not all patients respond to VEGF or anti-VEGF treatments. For example, about 30% of patients with various retinopathies do not respond well to anti-VEGF treatments to stop pathological angiogenesis. Some patients further develop resistance to the current anti-VEGF treatments months after the treatments started. Peptide Lv is a molecule much smaller than VEGF. Its amino acid sequence is different from VEGF, so if patients do not respond to VEGF or anti-VEGF treatments, patients may respond to peptide Lv or anti-peptide Lv (anti-peptide Lv antibody) treatments. Peptide Lv and VEGF have synergistic effects, so peptide Lv or anti-peptide Lv may further enhance the efficacy of current existing VEGF or anti-VEGF therapies.

In addition to its angiogenic properties, peptide Lv also promotes L-VGCC currents, which are important regulators in cardiac and neuronal function. In some forms of heart failure and cardiac ischemia (lack of blood flow), they are associated with reduced cardiac contractility and perfusion. Peptide Lv can promote cardiac angiogenesis and vasodilation (for providing more blood flow) and enhance cardiac L-VGCC currents (thus increasing cardiac contractility). These unique dual effects will benefit the ischemic heart failure patients. Nearly 12% (1 million) of all US patients with coronary artery diseases have diffuse vascular problems (small vessel diseases), and the majority of them suffer from microvascular dysfunction, so the traditional treatments will not be sufficient to treat such conditions. If left untreated, small vessel diseases can lead to heart failure, myocardial infarction, and cardiac arrest. Thus, peptide Lv, with its angiogenic and vasodilation properties, will be the important new agent in treating these coronary artery diseases.

### SUMMARY OF THE INVENTION

An embodiment of the invention is directed to the administration of the composition containing a portion of the peptide Lv to a subject to promote angiogenesis. Evidence of peptide Lv as an angiogenic agent is shown by its ability to promote endothelial cell proliferation and intra-ocular neovascularization.

In a further embodiment of the invention, the interaction between peptide Lv and VEGFR2 represents a novel pathway regulating angiogenesis and cardiac function.

Another embodiment of the invention is directed to the use of anti-Lv antibodies for reducing angiogenesis and dampening L-VGCC activities.

An additional embodiment of the invention is directed to the composition and use of an artificially modified peptide Lv, the inverso D-peptide Lv, in which the first amino acid at the N-terminal is inverted from the natural L-isomer to D-isomer. The D-peptide Lv shows a similar efficacy in promoting endothelial cell proliferation as the natural peptide Lv.

### BRIEF DESCRIPTION OF THE DRAWINGS

FIGS. 1A, 1B, 1C, 1D, 1E, 1F and 1G show that peptide Lv enhances L-VGCC currents and protein expression in cultured embryonic cardiomyocytes and anti-peptide Lv antibody (a-peptide Lv) has an antagonist effect;

FIGS. 2A and 2B show that peptide Lv mediated enhancement of L-VGCC currents is blocked by VEGFR2 antagonist;

FIGS. 3A, 3B, 3C, 3D and 3E show that treatment of cells with peptide Lv elicits a significant increase of phosphorylated ERK levels and treatment with a kinase inhibitor in the presence or absence of peptide Lv decreased the current densities of L-VGCCs;

FIGS. 4A, 4B, 4C and 4D show that the endogenous agonist VEGFa mimics the effect of peptide Lv and enhances L-VGCC activity in chicken embryonic cardiomyocytes;

FIG. 5 shows that peptide Lv stimulates the cell proliferation of human umbilical vein endothelial cells (HUVECs) in a dose-dependent manner;

FIGS. 6A, 6B and 6C show that peptide Lv elicits vasodilation, promotes endothelial migration for wound healing, and enhances VEGF's effect on endothelial proliferation;

FIGS. 7A and 7B show that peptide Lv promotes vascular growth and FIG. 9 anti-peptide Lv antibody (anti-peptide Lv) dampens the vascular growth;

FIG. 8 shows that anti-peptide Lv antibody prevents pathological angiogenesis and neovascularization; and

FIG. 9 shows that anti-peptide Lv antibody dampens VEGF-elicited endothelial cell proliferation in a dose-dependent manner.

### DESCRIPTION OF EXEMPLARY EMBODIMENTS

An embodiment of the invention is directed to a composition comprising a portion of peptide Lv. A further embodiment of the invention is directed to the administration of the composition containing a portion of the peptide Lv to a subject to promote angiogenesis. Evidence of peptide Lv as an angiogenic agent is shown by its ability to promote endothelial cell proliferation and intra-ocular neovascularization. Direct injections of peptide Lv into vitreous significantly promote new generation of blood vessels (neovascularization). Further, peptide Lv can promote wound healing by enhancing the proliferation and migration of endothelial cells. In freshly isolated porcine retinal arterioles, peptide Lv increases the diameter of the blood vessels thus showing the ability of peptide Lv as a vasodilator and potentially to decrease blood pressure, as well as to promote microvascular circulation.

Peptide Lv is a peptide that enhances L-VGCC activities in cone photoreceptors through increased expressions of both mRNA and protein levels. Because L-VGCCs are essential for neurotransmitter release and activity-dependent calcium influx in neurons, L-VGCCs are critical in gating neuronal activities and function. In the brain, L-VGCCs are required for cognitive functions, such as learning and memory. In the retina, L-VGCCs are essential for communications between neurons and transmitting the visual signals from retinal photoreceptors all the way to retinal ganglion cells and to the brain. The expression of peptide Lv can be detected in various brain regions, including hippocampus, cerebral cortex, and cerebellum, which are all important for cognitive function and various learning and memories. All the major neurons in the retina, especially retinal photoreceptors express peptide Lv. Thus, peptide Lv may play a role in enhancing the function of L-VGCCs in the retina and brain.

VEGFR2 (KDR/FLK-1) is a potential receptor for peptide Lv, which is an underlying mechanism for the augmentation of L-VGCCs by peptide Lv in the cardiomyocytes. Peptide Lv is able to activate the phosphorylation of VEGFR2 (KDR/FLK-1) and its downstream signaling, while inhibition of VEGFR2 (KDR/FLK-1) signaling blocked the actions of peptide Lv. Test results suggest that peptide Lv can serve as a novel activator of VEGFR2 through its augmentation of L-VGCCs in cardiomyocytes and its action on promoting proliferation of endothelial cells. Furthermore, peptide Lv has angiogenic properties and might play a regulatory role in the cardiovascular system.

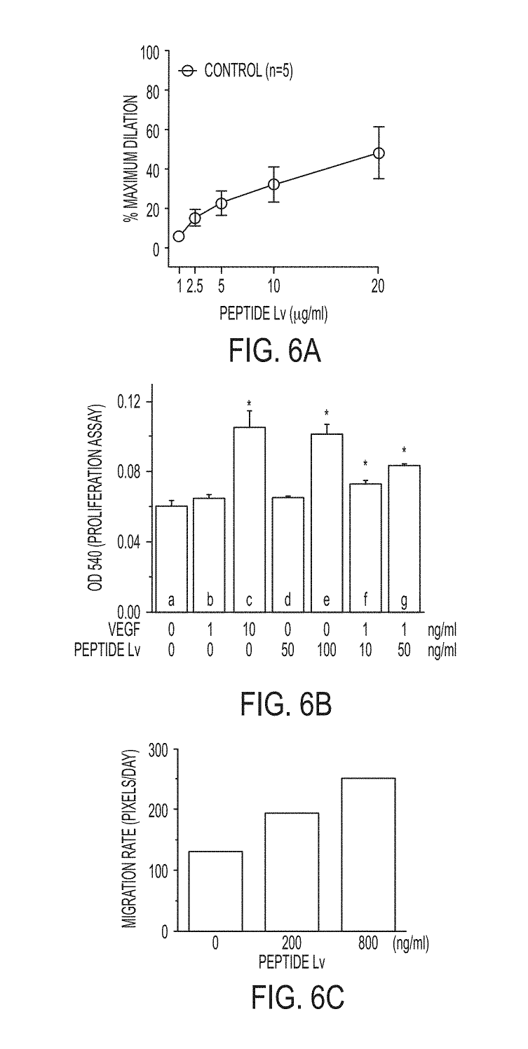

In the cardiovascular system, peptide hormones, such as somatostatin, angiotensin II, and natriuretic peptides (BNP and CNP), are known to be involved in the regulation of heart rate, cardiac contraction, and development. Several VEGF family members, including VEGFa, VEGFb, VEGFc, VEGFd, platelet-derived growth factor (PDGF), and placental growth factor (PLGF), can activate VEGFRs to regulate angiogenesis and lymphangiogenesis. Comparisons of the amino acid sequences of peptide Lv and the VEGF family have not revealed much homology. Through I-TASSER, a predicted secondary structure for peptide Lv was obtained and some similarities with VEGF members were found. Therefore, peptide Lv as an activator for VEGFR2 may differ from other VEGF family members when interacting with VEGFR2. While at low concentrations which neither VEGF nor peptide Lv would have any effect, adding both VEGF and peptide Lv together cause a significant enhancement of endothelial proliferation. Thus, peptide Lv has a synergistic action with VEGF (FIG. 6). However, the mechanism by which peptide Lv interacts with VEGFR2 and promotes VEGFR2 dimerization remains a question.

Activation of VEGFR2 signaling elicits a rise of intracellular calcium concentration through an increase of the intracellular calcium store-related proteins or the conductance of transient receptor potential (TRP) channels in various cell types. In cardiomyocytes, calcium influx through L-VGCCs triggers calcium release from intracellular calcium stores, activates calcium-dependent kinases, and leads to excitation-contraction coupling. The VEGF-PLCγ pathway is known to control cardiac contractility in the embryonic heart. Even though peptide Lv has been shown to increase L-VGCC currents and the expression of L-VGCCα1 subunits through the VEGFR2 signaling pathway in cardiomyocytes, whether L-VGCCs are involved in the VEGF-mediated calcium-induced intracellular calcium release needs future investigation. It is postulated that the augmentation of L-VGCC protein expression and currents by peptide Lv might lead to increased cardiomyocyte contractions, because treatments with other neuropeptides, such as endothelin-1 and angiotensin-II, have been shown to enhance calcium-dependent contraction in cardiomyocytes.

In summary, peptide Lv can interact with VEGFR2 and trigger receptor tyrosine kinase activity, as well as its downstream signaling pathway in cardiomyocytes. Peptide Lv enhances calcium entry by increasing L-VGCC currents through increasing the expression of L-VGCCα1 subunits. The interaction between peptide Lv and VEGFR2 represents a novel pathway regulating angiogenesis and cardiac function.

Another embodiment of the invention is directed to an antibody that is specific for a portion of the peptide Lv. The anti-peptide Lv antibody (anti-peptide Lv) decreases L-VGCC currents and blocks peptide Lv's actions. While L-VGCCs are essential in various cardiac, brain and retinal functions, dysregulation or hyperactivities of L-VGCCs can cause cardiac arrhythmia and epilepsy, and it may contribute to aging-related dementia. Thus, these antibodies are capable of reducing angiogenesis and dampening L-VGCC activities, and can be used for the treatment of diseases that involve pathological angiogenesis and lymphangiogenesis, L-VGCC hyperfunction, or neural dysfunction. These diseases include cancers, diabetic retinopathy, age-related macular degeneration, cardiac arrhythmia, various neuropathies, and the like.

An additional embodiment of the invention is directed to the use of an artificially modified peptide Lv, the inverso D-peptide Lv, in which the first amino acid at the N-terminal is inverted from the natural L-isomer to D-isomer. The D-peptide Lv shows a similar efficacy in promoting endothelial cell proliferation as the natural peptide Lv.

### WORKING EXAMPLES

### Chicken Embryonic Cardiomyocyte Culture

Fertilized eggs (Gallus gallus, Single Comb White Leghorns) were obtained from the Poultry Science Department, Texas A&M University (College Station, Tex., USA). All chicken embryos were maintained at 39° C.±0.5° C. Chicken hearts were harvested at embryonic day 12 (E12) and ventricular cardiomyocytes were dissociated and cultured on poly-D-lysine/collagen double-coated dishes (for biochemical and molecular biological assays) or coverslips (for electrophysiological recordings) as described previously. All cultures were maintained at 39° C.±0.5° C. with 5% CO₂. Cells were cultured for 2-3 days and subjected to treatments with vehicle, peptide Lv, or pharmaceutical blockers prior to Western blotting or electrophysiological recordings.

### Reverse Transcription PCR (RT-PCR)

Total RNA from mouse eyes, spleen, intestine, lung, and heart were isolated. One step RT-PCR amplification was used for detection of peptide Lv precursor and GAPDH mRNA expression as described previously.

### Western Blots and Trichloroacetic Acid (TCA) Precipitation

Treated or control cells were washed and lysed in radioimmunoprecipitation assay (RIPA) buffer, and proteins were denatured by mixing with 2× Laemmli sample buffer for 5 minutes at 95° C. Samples were separated on 10% sodium dodecyl sulfate-polyacrylamide gel electrophoresis (SDS-PAGE) gels and transferred to nitrocellulose membranes. The primary antibodies used in this study were pan L-VGCCα1, ERK1/2, di-phosphorylated ERK1/2, actin, phosphorylated PKC α/βII subunit, phosphorylated tyrosine, VEGFR2, and phosphorylated VEGFR2 at tyr1054/1059. Because chickens appear to express a single form of ERK (on the basis of molecular weight), the antibodies against ERK1/2 and pERK1/2 only label a single band on Western blots, while both antibodies label two bands in samples prepared from mammalian tissues. Blots were visualized by the appropriate secondary antibodies and an ECL detection system. For TCA precipitation, 100% (g/ml) TCA was added to each sample to achieve a final concentration of 20%. The samples were kept on ice for 4 hours then the precipitate was collected by centrifugation. Pellets were washed twice with acetone and completely air dried before addition of SDS sample buffer.

### Electrophysiological Recordings and Statistical Analysis

Ventricular cardiomyocytes were cultured for two days. Whole-cell patch-clamp recordings of L-VGCC currents were carried out from spontaneously pulsing cardiomyocytes. The external solution was (in mM): 145 TEACl, 9 BaCl₂, 0.5 MgCl₂, 5.5 glucose, 0.1 NiCl₂, and 5 HEPES, pH 7.4 with CsOH or TEAOH. The pipette solution was (in mM): 125 Cs acetate, 20 CsCl, 3MgCl₂, 10 EGTA, and 5 HEPES, pH 7.4 adjusted with CsOH. Currents were recorded at room temperature using an A-M Systems model 2400 patch-clamp amplifier (Sequim, Wash., USA). Signals were low-pass filtered at 2 kHz and digitized at 5 kHz with Digidata 1440A interface and pCLAMP 10.0 software (Molecular Devices, Sunnyvale, Calif., USA). Cardiomyocytes were held at −40 mV, and Ba²⁺ currents were recorded immediately after whole-cell patches were formed by gentle suctions. Current-voltage (I-V) relationships were elicited from a holding potential at −40 mV using 200 ms steps (5 seconds between steps) over a range from −60 to +60 mV in 10 mV increments. The current densities were calculated by dividing the current amplitudes (pA) by membrane capacitances (pF).

Co-Immunoprecipitation and Proteomics with Mouse Brains

Adult mouse whole brains were collected and lysed in 8 ml immunoprecipitation buffer containing a protease inhibitor cocktail. The cell lysate was cleaned with 1 ml sepharose 4B resin prior to incubation with peptide Lv antibody conjugated sepharose 4B. A 10 μg custom-made rabbit polyclonal antibody was immobilized in 40 μl AminoLink Plus Coupling Resin by following the manufacturer's protocol. The lysate and antibody were incubated for 5 hours at 4° C. The resins were washed three times with immunoprecipitation buffer and twice with PBS. Samples were resolved on 12% SDS-PAGE gels and stained with Coomassie brilliant blue R-250. Four major bands ranging from 40 to 200 kD were carefully excised and subjected to in-gel digestion and matrix-assisted laser desorption/ionization-time of flight (MALDI-TOF) mass spectrometry. Mass spectrometry and database analysis were performed in the Laboratory of Biological Mass Spectrometry, Texas A&M University.

### Co-Immunoprecipitation (Co-IP)

Eight chicken embryonic hearts (E18) were collected and homogenized in 4 ml of immunoprecipitation buffer. Samples were rotated at 4° C. for 2 hours to solubilize membrane proteins. Samples were then centrifuged to remove cell debris, and a small portion of the supernatant was taken for protein quantification analyses and for a SDS-PAGE gel subsequently stained with Coomassie brilliant blue R-250. The rest of the supernatant was cleaned with Protein Agarose beads followed by incubating the beads with 10 μl of the antibody (anti-peptide Lv or anti-VEGFR2) for 3 hours. Because both anti-peptide Lv and anti-VEGFR2 antibodies were derived from the rabbit, rabbit IgG was used as the negative control. Any protein fractions that could co-IP with rabbit IgG, as well as anti-peptide Lv or anti-VEGFR2 antibody, would be considered to be non-specific protein targets of peptide Lv. After incubation, 20 μl of Protein A-agarose was added to each tube and incubated for another 1.5 hours. The beads were collected, washed, and processed for Western blotting analysis of peptide Lv and VEGFR2. Western blots were visualized as described previously. For some IPs, antibody preparatory and immunoprecipitation kits were used to remove heavy and light chain interference. All co-IPs were repeated three times.

Tetrazoliumdye 3-(4,5-dimethylthiazol-2-yl)-2,5-diphenyltetrazolium bromide (MTT) Colorimetric Assay for Cell Proliferation

Human umbilical vein endothelial cells (HUVECs) and Human retinal endothelial cells (RECs) were seeded onto 24-well plates in endothelial cell growth medium and allowed to adhere overnight. The culture media were exchanged to opti-MEM for 45 minutes. VEGF or Peptide Lv at various concentrations were added to the cells, and the cells were continuously incubated for another 48 hours (HUVECs) or 96 hours (RECs). Opti-MEM with 20% FBS alone acted as the negative control. The proliferation of HUVECs was determined by the MTT assay following the manufacturer's protocol. In brief, cells were incubated with the MTT solution (1.2 mM final concentration) for 4 hours at 37° C. Then DMSO was added to a final concentration of 50% in order to break the plasma membrane, and the absorbance at 540 nm was measured by a spectrophotometer.

### Assessment of Vasodilation Function

All animal procedures were performed in accordance with the Association for Research in Vision and Ophthalmology (ARVO) Statement for the Use of Animals in Ophthalmic and Vision Research and were approved by the Scott and White Institutional Animal Care and Use Committee. Pigs of either sex (age range, 8-12 weeks; weight, 8-12 kg) purchased from Real Farms (San Antonio, Tex.) were sedated with Telazol (4.4 mg/kg, intramuscularly), anesthetized with 2-4% isoflurane, and intubated. The eyes were enucleated and immediately placed into a moist chamber on ice. The anterior segment and vitreous body were removed carefully under a dissecting microscope. The posterior segment or eyecup was placed in a cooled dissection chamber (6° C.) containing a physiological salt solution (PSS; in mM: NaCl 145.0, KCl 4.7, CaCl₂ 2.0, MgSO₄ 1.17, NaH₂PO₄ 1.2, glucose 5.0, pyruvate 2.0, EDTA 0.02, and MOPS 3.0) with 1% albumin. Retinal arterioles (<80 μm in diameter) were carefully dissected out and then transferred for cannulation to a Lucite vessel chamber containing PSS-albumin solution equilibrated with room air at ambient temperature. One end of the arteriole was cannulated using a glass micropipette (tip outer diameter of 30-40 μm) filled with PSS-albumin solution, and the outside of the arteriole was securely tied to the pipette with 11-0 ophthalmic suture. The other end of the vessel was cannulated with a second micropipette and also secured with suture. After cannulation, the vessel and pipettes were transferred to the stage of an inverted microscope (Olympus CKX41) coupled to a video camera (Sony DXC-190, Labtek, Campbell, Calif.) and video micrometer (Cardiovascular Research Institute, Texas A&M University System Health Science Center, College Station, Tex.) for continuous measurement of the internal diameter throughout the experiment. The cannulating pipettes were connected to independent pressure reservoirs. By adjusting the height of the reservoirs, the vessel was pressurized to 55 cmH₂O intraluminal pressure without flow. This level of pressure was used based on pressure ranges that have been documented in retinal arterioles in vivo and in the isolated, perfused retinal microcirculation. After developing resting vascular tone (˜40 min), the concentration-dependent response of isolated vessels to peptide Lv, (1 to 20 μg/ml) was examined and the diameter changes were recorded. At the end of the experiments, the vessels were maximally dilated with sodium nitroprusside (0.1 mM) in the bath containing zero calcium. These maximum dilations were used to normalize the peptide Lv response and expressed as % maximum dilation.

### In Ovo (In Vivo) Chicken Chorioallantoic Membrane (CCM) Assays for Angiogenesis and Vasculogenesis

The in ovo (in vivo) CCM assay is an effective test to determine whether exogenous molecules promote blood vessel growth in length (angiogenesis) or forming new blood vasculature (vasculogenesis). Using shell-less chicken embryo cultures, two coverslips coated with PBS (vehicle), peptide Lv (5 μg/per coverslip), or anti-peptide Lv (1 μg/per coverslip) were placed on the top of the CCM of each embryo at embryonic day 7 (E7) or E8. At E11 or E12, photographs of the CCM areas with coverslips were taken, and the vasculature areas and vessel lengths were analyzed.

### Intra-Ocular Injection and Retinal Vessel Staining

The male and female C57BL/6J mice were originally purchased from Harlan (Houston, Tex., USA). All animal experiments were approved by the Institutional Animal Care and Use Committee of Texas A&M University and were performed in compliance with the ARVO Statement for the Use of Animals in Ophthalmic and Vision Research. Mice were housed under temperature and humidity-controlled conditions with 12:12 hour light-dark cycles. All mice were fed with laboratory chow and water ad libitum. At postnatal day 7 (P7), young mice were anesthetized with isoflurane, and the intravitreal injections into the vitreous of the eye with PBS (vehicle buffer) or peptide Lv were performed using a Hamilton syringe. One eye from each mouse was injected with peptide Lv (at 2 μg/μl) at 0.5 or 1 μl, and the other eye was either injected with PBS or without injection. At P13, mice were sacrificed, and the eyes were fixed with Zamboni's fixative for 2 hours at 4° C. The retinas were isolated and stained overnight at 25° C. with FITC-conjugated Isolectin B4 in PBS containing 0.1% TX-100 and 1 mM Ca²⁺. Following 2 hours of washes, retinas were cut on the peripheral edge and flat-mounted with the photoreceptor side down onto microscope slides in ProLong Antifade reagents. Images were taken at 5x magnification on a Zeiss Digital Imaging Workstation, and whole retinal images were stitched together with the Image Composite Editor.

### Endothelial Migration (Wound Healing) Assay

The HUVECs were cultured on coverslips to 70% confluence. A “scratch” gap was generated using a 200 μl-pipette tip. Cultures were treated with either PBS (control) or peptide Lv (200 or 800 ng/ml). Photos were taken with the Olympus IX 71 inverted microscope at various time post-scratch, and cell migration distance from the scratch mark was measured (in pixels) using Adobe Photoshop software, and the migration rate was calculated as pixels per day (24 hours).

### Assessment of Pathological Angiogenesis Using the Mouse Oxygen-Induced Retinopathy (OIR) Model

The mouse OIR model has been widely used as a model for pathological angiogenesis. At postnatal day 7 (P7), young newborn pups were kept in a hyperoxia environment (75% oxygen) until P12, and after P12, the pups were returned back to the normal room air (normoxia). Under hyperoxia (from P7 to P12), there is blood vessel loss in the retina. After P12 to P17 under normal room air (normoxia), retina will have robust re-growth of blood vessels, including angiogenesis, vasculogenesis, and vessel proliferation, so the OIR model is used to induce pathological angiogenesis. Testing molecules or vehicle (control) are injected into the eyes at P12 immediately upon returning to the normal room air, and mice are sacrificed with the retinas excised at P17 for further analyses. The retinal areas that are lacking vasculature (“avascular area”) are quantified. If a molecule promotes angiogenesis, the avascular retinal area of the molecule-injected eye will be less than the control eye (injected with vehicle). If a molecule prevents angiogenesis, the avascular retinal area of the molecule-injected eye will be larger than the control eye. At P12, one eye from each young mouse was injected with PBS (vehicle), while the other eye was injected with anti-peptide Lv (2 μg/μl). At P17, mice were sacrificed, and the eyes were fixed with Zamboni's fixative for 2 hours at 4° C. The retinas were isolated and processed for retinal vasculature staining as described above, and the retinal area that was lacking vasculature (“avascular area”) were quantified.

### RESULTS

### Peptide Lv Enhances L-VGCC Activities in Time- and Dose-Dependent Manners in Cardiomyocytes

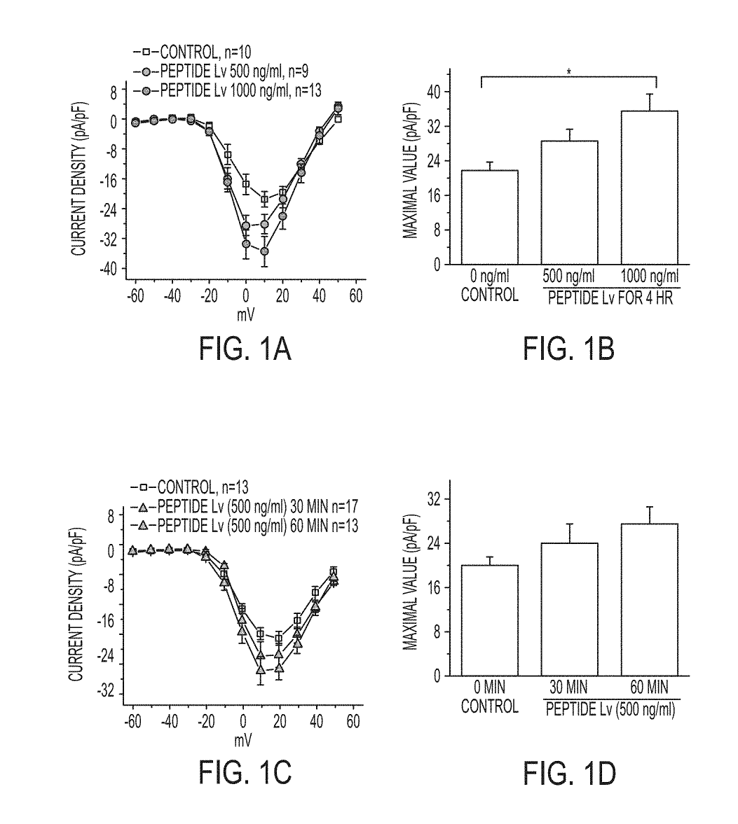

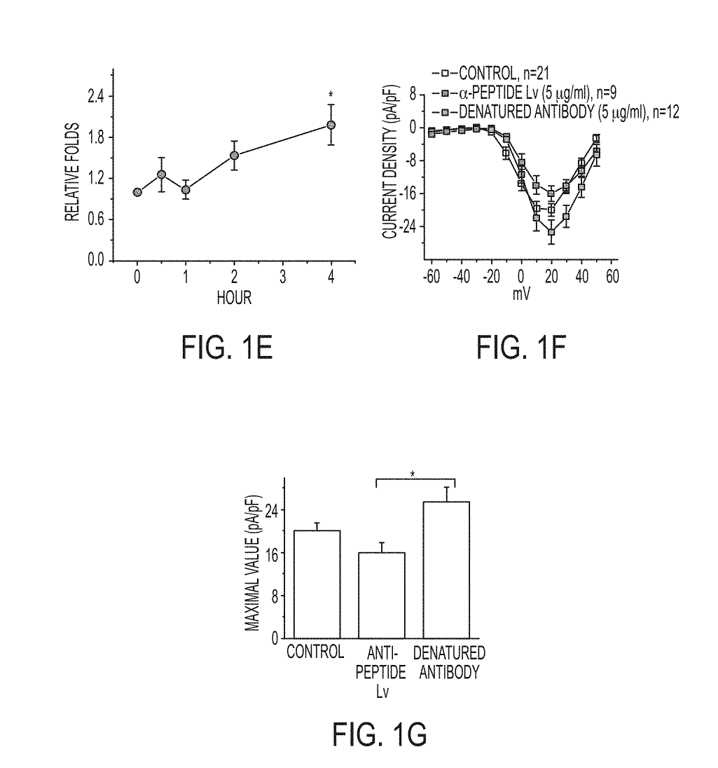

It has been shown that peptide Lv enhances the L-VGCC currents in retinal photoreceptors in time- and dose-dependent fashions. Since the L-VGCCs are essential in the excitation-contraction coupling of cardiomyocytes, it was postulated that peptide Lv might also regulate the L-VGCCs in cardiomyocytes similar to its action in the photoreceptors. FIG. 1 shows that peptide Lv enhances L-VGCC currents and protein expression in cultured embryonic cardiomyocytes; FIGS. 1A and 1B show that the augmentation of L-VGCC currents by peptide Lv is concentration-dependent. Cardiomyocytes were dissociated and cultured at E12, and L-VGCC currents were recorded at E14. Cultures were treated with 0, 500, and 1000 ng/ml of synthetic peptide Lv for 4 h prior to electrophysiological recordings. There was a stepwise increase in the L-VGCC current densities with 1000 ng/ml peptide Lv being significantly higher. FIGS. 1C and 1D shows that the effect of peptide Lv on LVGCCs in cardiomyocytes is time-dependent. Cardiomyocytes were treated with synthetic peptide Lv (500 ng/ml) for 0, 30, or 60 min prior to electrophysiological recordings. FIG. 1E shows that peptide Lv enhances L-VGCCα1 expression. Treatment with synthetic peptide Lv (500 ng/ml) for 4 h in cultured cardiomyocytes (E12+2) elicited a significant increase of L-VGCCα1 expression (1.98±0.29 folds; * indicates a significant difference at p<0.05). FIGS. 1F and 1G show that an anti-peptide Lv antibody (a-peptide Lv) decreases L-VGCC currents. Cultures were treated with peptide Lv antibody or heat-inactivated peptide Lv antibody (5 μg/ml; denatured antibody) for 24 h prior to electrophysiological recordings. Treatment with the peptide Lv specific antibody decreased the maximal current density of L-VGCCs compared to the L-VGCCs recorded from the control or cardiomyocytes treated with denatured antibody.

Cultured embryonic cardiomyocytes were treated with a synthetic peptide Lv for 4 hours at 500 ng/ml or 1000 ng/ml followed by patch-clamp electrophysiological recordings of L-VGCC currents. At 1000 ng/ml, peptide Lv elicited significantly higher L-VGCC currents (e.g., see FIGS. 1A and 1B). Treatment with peptide Lv for only 30 or 60 minutes quickly elicited an increase of L-VGCC currents (e.g., see FIGS. 1C and 1D), which was in part through an increase of protein expression of the pore-forming L-VGCCα1 subunits (e.g., see FIG. 1E). Because the mRNA of peptide Lv is present in various tissues, including the heart, eye, and various brain areas, the existence of endogenous functional peptide Lv needs to be verified. If a functional peptide Lv is expressed in the heart, application of an antibody specifically against peptide Lv would affect L-VGCCs by antagonizing the action of endogenous peptide Lv in embryonic cardiomyocytes. Cultured embryonic cardiomyocytes were treated with a specific antibody against peptide Lv for 18-22 hours prior to patch-clamp recordings. Cardiomyocytes treated with the anti-peptide Lv antibody (a-peptide Lv) showed decreased L-VGCC currents, while in contrast, cardiomyocytes treated with a denatured anti-peptide Lv antibody did not have effect on L-VGCC currents (FIG. 1F). These results demonstrated that exogenous peptide Lv augments the L-VGCCs by enhancing the expression of L-VGCCα1 subunits, and blocking the endogenous peptide Lv dampens the L-VGCC currents, indicating a functional role of peptide Lv in cardiomyocytes.

### Identification of VEGFR2 (KDR/FLK-1) as a Binding Partner for Peptide Lv

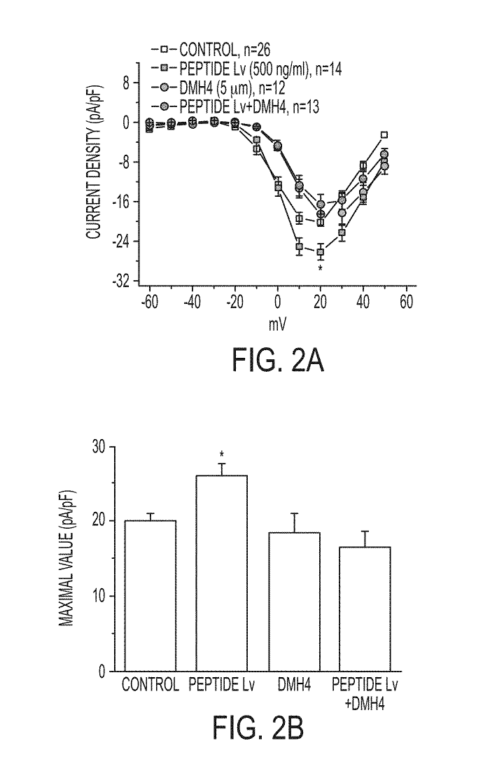

A proteomics approach was used to identify potential receptors or binding partners in order to determine the underlying molecular mechanisms of peptide Lv on L-VGCCs in both photoreceptors and cardiomyocytes. Since the mouse brain also expresses peptide Lv abundantly and yields more tissue than the heart, a mouse whole brain preparation with co-immunoprecipitation (co-IP) was used followed by a SDS-PAGE and mass spectrometry (MS) analysis to determine the potential receptor candidates for peptide Lv. Excluding the cytoskeleton chaperone proteins (myosin, clathrin heavy chain, and tubulin), there were three potential receptor-like candidates, including KDR protein (VEGFR2/KDR/FLK-1) (Access No. EDL37891), Fc receptor-like B (Access No. NP_001025155), and vomeronasal type-1 receptor (Access No. NP_035814). The vomeronasal type-1 receptor is a G-protein coupled pheromone receptor mainly located in the olfactory bulb. The Fc receptor-like B is a member of the Fc receptor family that is involved in phagocytosis, antibody-dependent cell cytotoxicity, and transcytosis. The KDR/FLK1 protein, also known as the vascular endothelial growth factor receptor 2 (VEGFR2), belongs to the tyrosine kinase (TK) receptor family. Further proteomics analysis indicated that VEGFR2 (KDR/FLK1) and vomeronasal type-1 receptor were the possible candidate receptors for peptide Lv. To determine which receptor interacted with peptide Lv, co-IP assays were employed, and an interaction between peptide Lv and VEGFR2 in the chick hearts was found. Using the anti-peptide Lv antibody, a protein near 250 kD was coimmunoprecipitated from the E18 chicken hearts, which was detected with the anti-VEGFR2 antibody. While the anti-VEGFR2 antibody was used to co-immunoprecipitate proteins, a protein near 6 kD was detected with the anti-peptide Lv antibody. In both cases, the presence of VEGFR2 or peptide Lv was not detected using the rabbit IgG for co-IP. In cultured cardiomyocytes, treatment with a selective VEGFR2 inhibitor, DMH4, was able to block the augmentation effect of peptide Lv on L-VGCC currents (FIGS. 2A and 2B), but DMH4 by itself did not affect the LVGCCs, indicating that the action of peptide Lv on L-VGCCs was in part through the VEGFR2. Hence, VEGFR2 (KDR/FLK1) is a potential receptor for peptide Lv.

### Peptide Lv Stimulates VEGFR2 Autophosphorylation in Cardiomyocytes

Since VEGFR2 belongs to a tyrosine kinase receptor family that is subjected to autophosphorylation of tyrosine residues after the receptor is activated via ligand binding, whether peptide Lv could elicit tyrosine phosphorylation of VEGFR2 was examined next. Among the four tyrosine phosphorylation sites on VEGFR2, tyr1054/1059 phosphorylation is required for maximal kinase activation and is considered a prerequisite for activating VEGFR2 signaling. Because the amino acid sequences near tyr1054/1059 of VEGFR2 are highly conserved across the chicken, mouse, and human, the specific antibody against phosphorylated VEGFR2 at tyr1054/1059 (pVEGFR2-tyr1054/1059) was used to detect VEGFR2 autophosphorylation. A p-tyr-100 antibody that commonly used to detect phosphorylated tyrosine residues, was employed to assess the activation of tyrosine kinases. Cultured chicken embryonic cardiomyocytes were treated with peptide Lv in the presence or absence of VEGFR2 inhibitor DMH4. Peptide Lv was found to enhance phosphorylation of VEGFR2 (pVEGFR2-tyr1054/1059) and phosphorylated tyrosine in a manner sensitive to DMH4 inhibition. Treatment with peptide Lv was able to elicit tyrosine autophosphorylation of VEGFR2 (pVEGFR2-tyr1054/1059) within 30 minutes, and this phosphorylation was time-dependent with a maximal activation at 2 hours. Hence, VEGFR2 is a candidate receptor for peptide Lv.

### Peptide Lv Activates Downstream Signaling Molecules of VEGFR2 in Cardiomyocytes

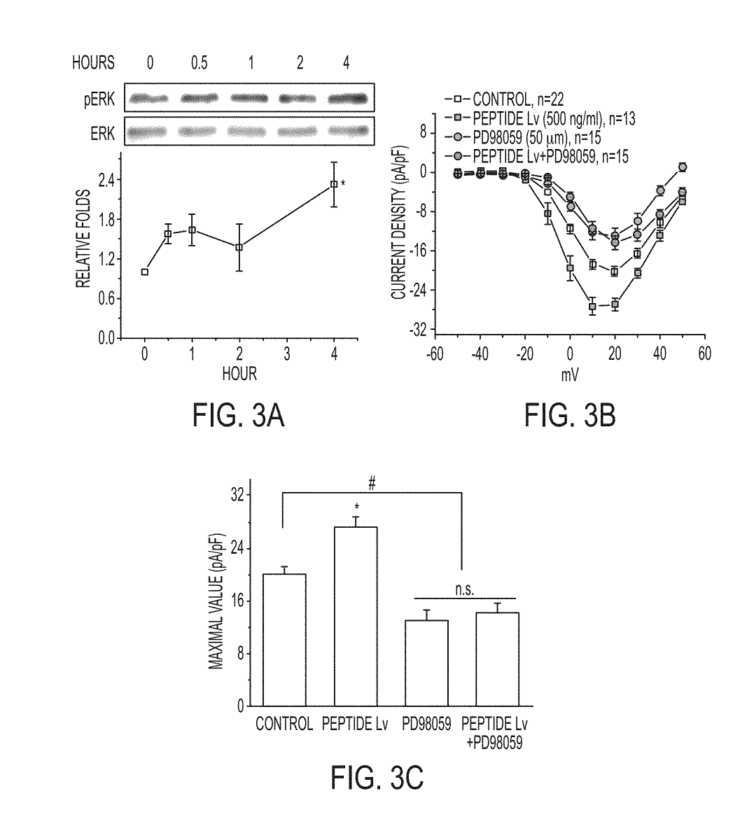

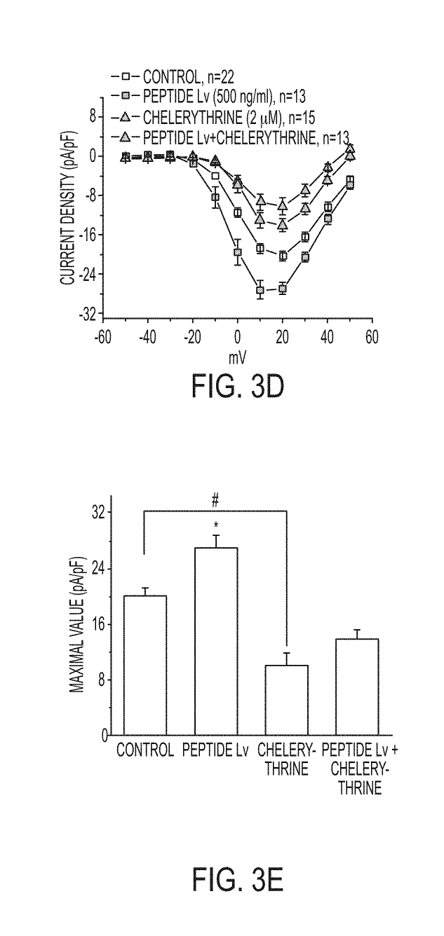

Activation of VEGFR2 triggers several downstream signaling cascades, including phosphoinositide phospholipase C (PLCγ), phosphoinositide 3-kinase (PI3K), and mitogen-activated protein kinase (MAPK) and extracellular signal-regulated kinase (ERK) pathways. Phosphoinositide phospholipase C (PLC) participates in phosphatidylinositol 4,5-bisphosphate (PIP2) metabolism and generates inositol triphosphate (IP3) and diacylglycerol (DAG), in which DAG further serves as an activator of protein kinase C (PKC). In both porcine aortic endothelial cells and cultured cardiomyocytes, activation of VEGFR2 causes phosphorylation and activation of ERK1/2. Since PKC and ERK are downstream of VEGFR2, whether these signaling molecules would respond to peptide Lv was examined in a series of experiments. In cultured cardiomyocytes, treatment of peptide Lv for 4 hours increased the phosphorylation levels of ERK (pERK, FIG. 3A) and PKC (pPKCα/β). While the general G-protein coupled receptor (GPCR) inhibitor SCH202676 did not inhibit the effects of peptide Lv on pERK and pPKCα/β, the VEGFR2 inhibitor DMH4 did, indicating that peptide Lv is able to elicit activation/phosphorylation of the downstream signaling targets of VEGFR2, such as the ERK and PKC. Treatment with PD98059 (50 μM), an inhibitor of MEK1 upstream of ERK, decreased the peptide Lv augmentation of L-VGCC currents (FIGS. 3B and 3C), as did the PKC inhibitor chelerythrine (2 μM, FIGS. 3D and 3E). Inhibition of PKC or MEK1-ERK signaling blocked the effect of peptide Lv on L-VGCC activity. Hence, the action of peptide Lv on L-VGCC augmentation is suggested to be mediated by the VEGFR2 binding, which further activates downstream signaling, including PKC and ERK.

### The VEGF Signaling Pathway Increases L-VGCC Activities in the Cardiomyocytes

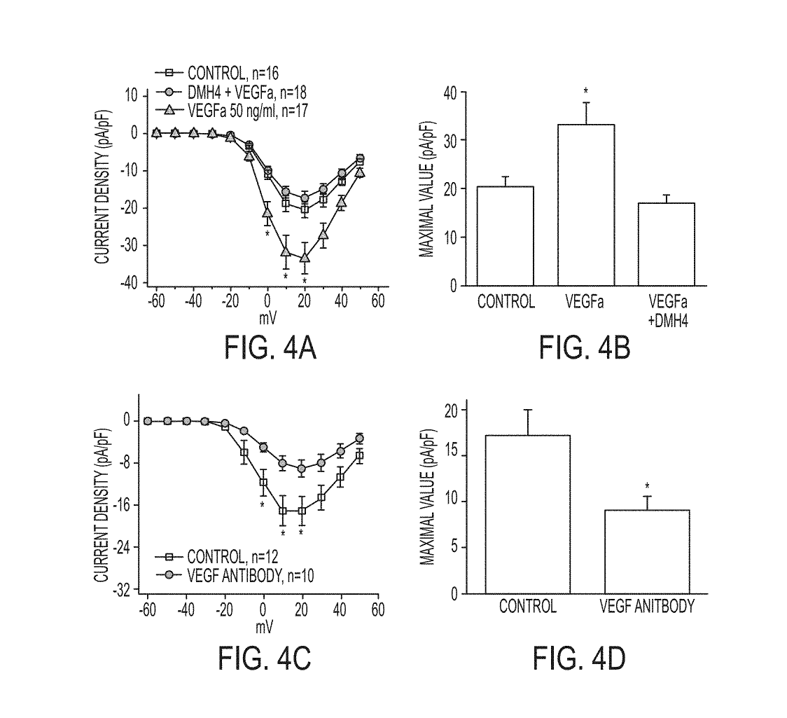

As discussed earlier, peptide Lv augments L-VGCC activities through the VEGFR2 signaling pathway in chicken cardiomyocytes. While both VEGFR2 and VEGFa (an endogenous VEGFR2 agonist) are present in cultured embryonic or neonatal cardiomyocytes, thus far, there is no report on the increase of L-VGCCs following the direct activation of VEGFR in cardiomyocytes. Treatment with VEGFa (50 ng/ml) for 4 hours significantly increased L-VGCC currents (FIGS. 4A and 4B). Treatment with the VEGFR2 antagonist (DMH4,5 μM) blocked the augmentation of L-VGCCs by VEGFa (FIGS. 4A and 4B). Treatment with a specific antibody against VEGFa (VEGF antibody) significantly decreased the L-VGCCs activity in cultured embryonic cardiomyocytes (FIGS. 4C and 4D), indicating that endogenous VEGF signaling is involved in the regulation of L-VGCCs in embryonic cardiomyocytes. These data support the notion that activation of VEGFR2 can augment the L-VGCCs in cardiomyocytes, which further supports that peptide Lv mimics endogenous VEGF and increases L-VGCC currents in cardiomyocytes through activation of VEGFR2 signaling.

### Peptide Lv Stimulates the VEGFR2 Activation in the Human Umbilical Vein Endothelial (HUVE) Cells

The interaction between peptide Lv and VEGFR2 was also tested in cultured human umbilical vein endothelial cells (HUVECs) to verify that the action of peptide Lv to activate VEGFR2 was not solely in chicken cardiomyocytes. HUVECs were chosen due to the high expression of VEGFR2 in vascular endothelial cells and the identified role of VEGFR2 signaling in angiogenesis. The VEGFR2 was co-immunoprecipitated (co-IP) with the anti-peptide Lv antibody (a-peptide Lv) but not with the rabbit IgG from cultured HUVECs, which was similar to the co-IP result in embryonic chick hearts. To further validate if peptide Lv could also activate VEGFR2 (measured as pVEGFR2) in HUVECs as in chicken cardiomyocytes, cultured HUVECs were treated with 500 ng/ml peptide Lv for 0, 30, or 60 minutes before cells were harvested for Western immunoblotting. Antibodies against pVEGFR2 (tyr1054/1059), total VEGFR2, total ERK1/2, and pERK1/2 were used in the Western blots. Treatments with peptide Lv in cultured HUVECs increased both pVEGFR2 and pERK1/2 levels. Therefore, peptide Lv is able to activate VEGFR2 and its downstream signaling pathway in cultured human endothelial cells, as well as in chicken cardiomyocytes.

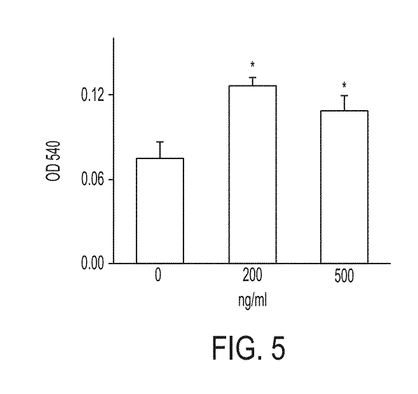

VEGF signaling regulates the proliferation, migration, and survival of endothelial cells and thus promotes angiogenesis. Mutations of VEGFR2 result in deficits in blood-island formation and angiogenesis, which is lethal during embryonic development. In response to angiogenic stimuli, endothelial cells proliferate, migrate, and coalesce to form a primitive vascular system and further recruit smooth muscle cells to give rise to mature blood vessels. Since peptide Lv activates VEGFR2 and its downstream signaling in both cardiomyocytes and vascular endothelial cells, it is possible that peptide Lv might enhance the proliferation of endothelial cells thus promoting angiogenesis. The HUVECs were treated with peptide Lv (200 or 500 ng/ml) or vehicle for 48 h, and then subjected to the tetrazolium dye (MTT) colorimetric assay to assess cell proliferation. The cells treated with peptide Lv (both at 200 and 500 ng/ml) had a significant increase in the color absorbance at 540 nm (FIG. 5), indicating that peptide Lv stimulates the proliferation of endothelial cells.

### Peptide Lv Elicits Vasodilation

The proper function of an organ requires adequate supply of blood flow (e.g., oxygen and nutrients) to the cells and tissues. The small arterioles (<100 μm in diameter) play a critical role in the control of blood flow by contracting or relaxing vascular smooth muscle, which causes vasoconstriction or vasodilation, respectively. Therefore, the dilation of arterioles becomes important in terms of mainlining tissue blood flow during ischemia or hypoxia. VEGF is known to be released during tissue hypoxia or ischemia, and it can cause vasodilation to increase blood flow. To determine whether peptide Lv also exhibits vasomotor activity (vasodilation ability) like VEGF, porcine retinal arterioles were freshly isolated and then tested its reaction to peptide Lv. Treatment with peptide Lv dilates the arteriole (FIG. 6A), and its action is similar to that of VEGF as a potent vasodilator. The vasodilator property of peptide Lv can be utilized to reduce blood pressure and promote perfusion of microcirculation and optimally enhance tissue oxygenation and survival under stress.

### Peptide Lv and VEGF Work Synergistically in Promoting Endothelial Cell Proliferation

While peptide Lv is able to bind to VEGFR2 and elicit cell proliferation, it is not clear whether peptide Lv might compete with VEGF for VEGFR2, or have synergistic action with VEGF. The human retinal endothelial cells (RECs) were cultured and treated with VEGF with or without peptide Lv for up to 96 hr. The MTT assays were carried out to measure the proliferation of RECs. Treating RECs with low concentration of VEGF (at 1 ng/ml) or peptide Lv (at 50 ng/ml) alone did not elicit significant proliferation in RECs compared to the control (treated with vehicle buffer). However, treating RECs with combined VEGF (1 ng/ml) and peptide Lv (10 ng/ml or 50 ng/ml) significantly promoted the cell proliferation, compared to the control or VEGF (1 ng/ml) alone (FIG. 6B), indicating that peptide Lv and VEGF work synergistically in promoting endothelial proliferation. This is the first evidence showing the synergistic interaction of peptide Lv and VEGF in endothelial cells. This unique interaction highlights the potential of using peptide Lv and its antagonists to manipulate angiogenesis and tissue repair under pathophysiological conditions.

### Peptide Lv Elicits Angiogenesis and Neovascularization In Vivo and Promotes Wound Healing In Vitro

Even though peptide Lv promotes endothelial cell proliferation, which is the essential step for angiogenesis and neovascularization, it was not known whether peptide Lv is able to promote angiogenesis in animals. Direct injections of peptide Lv (2 μg/μl) into postnatal day 7 (P7) mouse eyes through intravitreal injections strikingly increased the generation of new blood vessels (neovascularization), compared to the control (no injection) or injection with PBS (vehicle buffer). This is the first direct evidence that peptide Lv is an angiogenic agent promoting neovascularization in living tissues. In cultured HUVECs with 70% confluence, a scratch gap was generated thus representing a scratch “wound injury”. Treatments with peptide Lv at 200 or 800 ng/ml significantly enhanced the endothelial cell migration rate compared to the control (treated with vehicle buffer), suggesting that peptide Lv is able to promote wound healing (FIG. 6C).

In Ovo (In Vivo) Chicken Chorioallantoic Membrane (CCM) Assays Demonstrate that Peptide Lv Promotes Angiogenesis and Vasculogenesis, while Anti-Peptide Lv Antibody (Anti-Peptide Lv) Dampens Angiogenesis and Causes Vaso-Obliteration

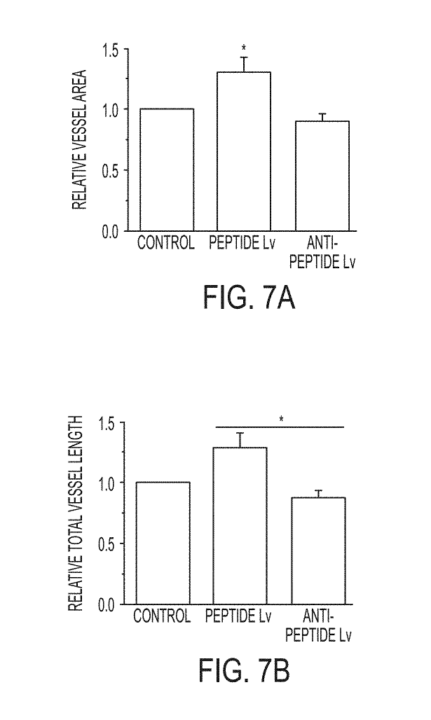

The in ovo (in vivo) CCM assay is an effective test to determine whether exogenous molecules promote blood vessel growth in length (angiogenesis) or forming new blood vasculature (vasculogenesis). Using shell-less chicken embryo cultures, two coverslips coated with either PBS (vehicle) or peptide Lv (5 μg/per coverslip) were placed on the top of the CCM of each embryo at embryonic day 7 (E7) or E8. At E11, more capillaries and small vascular branches are observed under the coverslips coated with peptide Lv. Hence, peptide Lv is able to promote angiogenesis and vasculogenesis in chicken embryos in ovo (FIGS. 7A, 7B). Using a similar experimental design with coverslips coated with either PBS or anti-peptide Lv (1 μg/per coverslip), it was found that the CCM areas covered with anti-peptide Lv for ˜4 days develop fewer capillaries and vascular branches compared to the areas covered with PBS-coated coverslips (control; FIGS. 7A, 7B)). Some small vessels under anti-peptide Lv showed signs of vaso-obliteration.

Intraocular Injections of Peptide Lv or Anti-Peptide Lv into Early Postnatal Young Mouse Eyes Demonstrate that Peptide Lv Promotes Angiogenesis and Vasculogenesis, but Anti-Peptide Lv Decreases Angiogenesis and Causes Vaso-Obliteration in the Retinal Vasculature During Normal Development

The retinal vasculature is undergoing angiogenesis and vasculogenesis during the first 3 weeks after birth. The postnatal day 7 (P7) mice were given a single intraocular injection into one eye with PBS (vehicle), and the other eye was injected with either peptide Lv (2 μg/μl), or anti-peptide Lv (2 μg/2 μl) for 7 days. At P14, the retinas were processed and stained for retinal vasculature. The eyes injected with peptide Lv have a significant increased retinal vasculature compared to the eyes injected with PBS or without injection, while the eyes injected with anti-peptide Lv shows signs of vaso-obliteration and decreased retinal vasculature.

### Anti-Peptide Lv Prevents the Re-Growth of New Blood Vessels in the Mouse Oxygen-Induced Retinopathy (OIR) Model

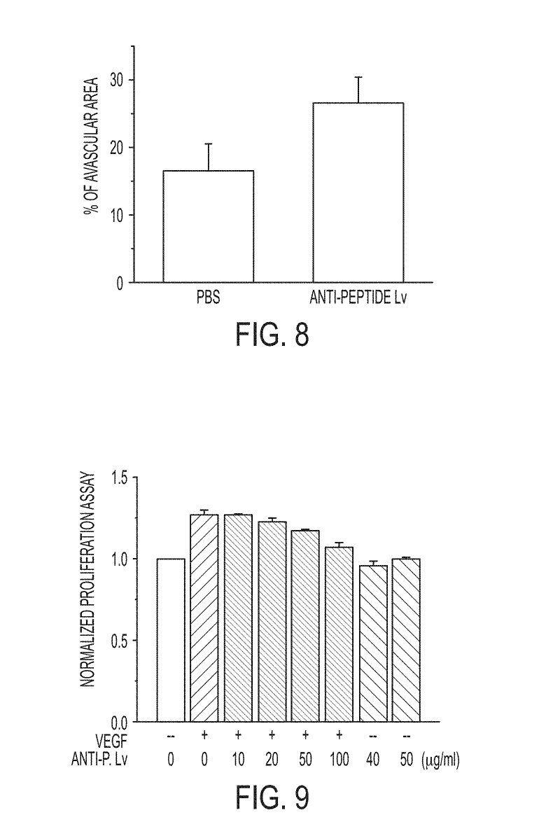

The mouse OIR model has been widely used as a model for pathological angiogenesis. At postnatal day 7 (P7), young newborn pups are kept in a hyperoxia environment (75% oxygen) until P12. Under hyperoxia (from P7 to P12), there is blood vessel loss in the retina. After P12 to P17 under normal room air (normoxia), retina will have robust re-growth of new vessels, which indicates the pathological angiogenesis and neovascularization. Molecules are injected into the eyes at P12, and the retinas are analyzed at P17. If a molecule promotes angiogenesis, the retinal area that is lacking vasculature (avascular area) will be less than the control eye (injected with vehicle). If a molecule prevents angiogenesis, the avascular area will be larger than the control eye. At P12, one eye from each young mouse was injected with PBS (vehicle), while the other eye was injected with anti-peptide Lv (2 μg/μl). The eyes injected with anti-peptide Lv had a larger avascular area compared to the eyes injected with PBS (FIG. 8). These data provide evidence that anti-peptide Lv is able to dampen pathological angiogenesis in vivo.

Modified Peptide Lv, the Inverso D-Peptide Lv, has a Similar Effect on Cell Proliferation Using the Tetrazoliumdye 3-(4,5-dimethylthiazol-2-yl)-2,5-diphenyltetrazolium bromide (MTT) Colorimetric Assay for Cell Proliferation

Human umbilical vein endothelial cells (HUVECs) were seeded onto 24-well plates in endothelial cell growth medium and allowed to adhere overnight. The culture media were exchanged to opti-MEM for 45 minutes. Peptide Lv or inverso D-peptide Lv (the very first amino acid at the N-terminal inverted from the natural L-isomer to D-isomer) at various concentrations (10, 50, 100 ng/ml) were added to the cells, and the cells were continuously incubated for another 48 hours (HUVECs). Opti-MEM with 20% FBS alone acted as the negative control. The cell proliferation of HUVECs was determined by the MTT assay as described previously. It was found that both peptide Lv and D-peptide Lv promote HUVEC proliferation equally.

### Anti-Peptide Lv Antibody has a Dose-Dependent Effect in Blocking VEGF-Elicited Endothelial Cell Proliferation in MTT Colorimetric Assays for Cell Proliferation

Using cultured HUVECs, cells were treated with various concentrations of anti-peptide Lv antibody (Anti-P. Lv) in the presence (+) or absence (−) of VEGF (20 ng/ml). Anti-peptide Lv antibody clearly dampens VEGF-elicited cell proliferation in a dose-dependent manner (FIG. 9).

While the present invention has been described in terms of certain preferred embodiments, it will be understood, of course, that the invention is not limited thereto since modifications may be made to those skilled in the art, particularly in light of the foregoing teachings.

What is claimed is:

1. A method of promoting angiogenesis in a subject, the method comprising administering to the subject a composition comprising a portion of peptide Lv.

2. The method of claim 1, wherein the administration of the composition stimulates VEGF signaling.

3. The method of claim 1, wherein the administration of the composition promotes vasodilation, angiogenesis, neovascularization, and L-VGCC's activities and function.

4. The method of claim 1, wherein the composition promotes cardiovascular and neurological function, wound healing, through promoting vasodilation, angiogenesis and lymphoangiogenesis, blood/lymphatic vessel repairing and new growth, and cardiac contractility and cardiac output, and enhancing cognitive function.

5. A method of treating disease comprising administering to a subject in need thereof a composition comprising an anti-peptide Lv antibody.

6. The method of claim 5, wherein the administration of the anti-peptide Lv antibody treats diseases that have pathological angiogenesis and lymphangiogenesis, L-VGCC hyperfunction, or neural dysfunction.

7. The method of claim 5, wherein the diseases comprise cancers, diabetic retinopathy, age-related macular degeneration, cardiac arrhythmia, and neuropathy.

8. The method of claim 5, wherein the anti-peptide Lv antibody blocks an effect of peptide Lv, VEGF, or VEGF receptors.

9. A composition for promoting the growth of new blood vessels, the composition comprising a modified peptide Lv, wherein the first amino acid at the N-terminal is inverted from an L-isomer to a D-isomer.

10. The composition of claim 9, wherein the first amino acid is a modified aspartic acid.
